(19) United States
(12) Patent Application Publication
(10) Pub. No.: US 2008/0187608 A1
(43) Pub. Date: Aug. 7, 2008

(54) ENRICHED FRACTIONS FROM CLARY SAGE FOR THE TREATMENT OF CANCER, CARDIOVASCULAR AND INFLAMMATORY DISEASES

(75) Inventors: Inderjit Kumar Dev, Durham, NC (US); Ven Subbiah, Greenville, NC (US)

Correspondence Address: PASSE' INTELLECTUAL PROPERTY, LLC, 1717 BRASSFIELD RD., RALEIGH, NC, 27614, US

(73) Assignee: SAVIPU PHARMACEUTICALS, Durham, NC (US)

(21) Appl. No.: 12/025,019

(22) Filed: Feb. 2, 2008

Related U.S. Application Data
(60) Provisional application No. 60/888,252, filed on Feb. 5, 2007.

(51) Int. Cl. A61K 36/537; A61P 35/04; A61P 29/00; A61P 9/00

(52) U.S. Cl. 424/746

(57) ABSTRACT

The present invention relates to the use of certain novel compositions from clary sage (Salvia sclarea) which are inhibitors of nuclear factor kappa B (NF-κB), oxidative stress (activation of Nerf2) and the activity of the endothelin receptor. In particular, it relates to useful enriched fractions and pharmaceutical compositions from clary sage for use in the treatment of cardiovascular and inflammatory diseases and for cancers susceptible to an inhibitor of oxidative stress (activation of Nerf2), NF-κB inhibitor and an endothelin receptor inhibitor. The present invention also relates to compositions from clary sage useful to inhibit cell proliferation and for the induction of apoptosis.

This application claims priority of the U.S. Application Ser. No. 60/888,252 filed on Feb. 5, 2007 and incorporated herein in its entirety by reference.

### FIELD OF THE INVENTION

This invention relates to the use of certain novel compositions from clary sage (Salvia sclarea) which are inhibitors of nuclear factor kappa B (NF-κB), oxidative stress (activation of Nerf2) and the activity of the endothelin receptor. In particular, it relates to useful enriched fractions and pharmaceutical compositions from clary sage for use in the treatment of cardiovascular and inflammatory diseases and for cancers susceptible to an inhibitor of oxidative stress (activation of Nerf2), NF-κB inhibitor and an endothelin receptor inhibitor. The present invention also relates to compositions from clary sage useful to inhibit cell proliferation and for the induction of apoptosis.

### BACKGROUND OF THE INVENTION

The U.S. Food and Drug Administration (FDA) has recently established a Botanical Drug Team as well as a set of guidelines for the development of complex botanical extracts as ethical pharmaceuticals with disease-related claims. Such changes in the FDA regulatory environment now allow for the development of botanicals at a much more rapid pace than new chemical entities or biologics, resulting in approval costs that may be reduced by an order of magnitude. Additionally, this new guidance provides for unique guarantees of market exclusivity for NDA botanicals as well as the acceptance of synergistic combinations of bioactives.

The synergistic components found in botanical mixtures represent a largely untapped source of new pharmaceutical products with novel and multiple mechanisms of action. Recent developments in plant biotechnology have created the tools to produce botanical mixtures at a level comparable to that of pure drug compounds, thus meeting the requirements of the FDA. Botanical drug products will ultimately compete along side conventional pharmaceuticals in the $300 billion global pharmaceutical marketplace.

Clary sage (Salvia sclarea) a native to the Mediterranean region, has been found growing in the wild, and has been used extensively in traditional medicines. Dried leaves of clary sage have been used mostly as a tea ingredient; occasionally in tablets, capsules and tincture. Traditionally, sage is used as a tonic, digestive, antiseptic, astringent, and antispasmodic. It has been used to reduce perspiration (e.g., night sweats), to re-stop the flow of milk, to treat nervous conditions (e.g., trembling, depression, and vertigo), dsymenorrhea, diarrhea, gastritis, sore throat, and insect bites usually in the form of a tea or infusion. Sage can serve as source of natural antioxidants. Clary sage oleoresin is also widely used in baked goods meats and meat products, and condiments and relishes. Sage oil has been extensively used in most categories of food products including alcoholic (e.g., vermouths and bitters) and nonalcoholic beverages, frozen dairy desserts, candy, baked goods, gelatins and puddings, meat and meat products, and condiments and relishes.

Clary sage occurs as an annual (rare), biennial (fairly common) or perennial (fairly common) open-pollinated herbaceous plant that has typical quadrangular stems, opposite leaves and verticillastrate inflorescence.

Clary sage is cultivated mainly for the production of essential oil, sclareol and sclareol derivatives. U.S. Pat. No. 3,060,172 describes a process for the isolation of sclareol from clary sage; U.S. Pat. Nos. 5,525,728 and 5,247,100 describe processes for the production of the sclareolide from sclareol.

A variety of other Salvia species including Salvia miltiorrhiza Bunge (Dan-Shen), Salvia przewalskii and Salvia yunnanensis have also been used for medicinal purposes (Chemical constituents in the roots of Salvia przewalskii Maxim, YaO-Xue-Bao, 38 (5): 354-357, 2003; Medicinal resources of Salvia yunnanensis, Zhong-Yao-Cai, 25(9): 628-629, 2002). Particularly, Dan-Shen has been used for centuries in traditional Chinese medicine for the treatment of coronary heart disease, particularly angina pectoris and myocardial infarction (Ji et al., Salvia Miltiorrhiza and Ischemic Diseases, Acta Pharmacol Sin 12: 1089-1094, December 2000). Danshen has also been used for anticoagulation, increase in urinary excretion, improved effects on uremic symptoms, and increased platelet aggregation and as an antifungal and antibacterial agent,

In the current invention, the inventors have found that the extracts prepared from clary sage roots (rhizomes) have potent activity against validated molecular targets for inflammatory diseases, respiratory and cardiovascular diseases and cancer. These molecular targets include nuclear erythroid 2 p45-related factor 2 (Nerf2), nuclear factor kappa B (NF-κB) and endothelin receptor A (ET_{A}). The endothelin receptors exist in various tissue and organs such as vessels, trachea and the like and their excessive stimulation can lead to circulatory diseases such as pulmonary arterial hypertension, acute and chronic heart failure, acute and chronic renal failure, atherosclerosis, cerebrovascular diseases and the like.

The family of NF-κB transcription factors comprises important regulatory proteins that impact virtually every feature of cellular adaptation, including responses to stress, inflammatory reactions, activation of immune cell function, cellular proliferation, programmed cell death (apoptosis), differentiation and oncogenesis (1). NF-κB regulates more than 150 genes, including cytokines, chemokines, cell adhesion molecules, and growth factors (2). It is therefore not surprising that diseases result when NF-κB—dependent transcription is not appropriately-regulated. NF-κB has been implicated in several pathologies, including certain cancers (e.g., Hodgkin's disease, breast cancer, and prostate cancer), diseases associated with inflammation (e.g., rheumatoid arthritis, asthma, inflammatory bowel disease (e.g., Crohn's disease and ulcerative colitis), alcoholic liver disease, non-alcoholic steatohepatitis, pancreatitis, primary dysmenorrhea, psoriasis, and atherosclerosis) and Alzheimer's disease. Inhibition of NF-κB also protects against chemotherapy-induced adverse effects including oral and GI mucositis, alopecia and neutropenia.

Oxidative stress contributes to the general decline in cellular functions that are associated with many human diseases including asthma, emphysema, Alzheimer disease, Parkinson disease, atherosclerosis, macular degeneration, degenerative retinal damage, rheumatoid arthritis, multiple sclerosis, muscular dystrophy, human cancers as well as the aging. To counteract oxidative stress, higher animals have developed elaborate mechanisms, including phase II detoxifying enzymes and antioxidant proteins. Induction of phase 2 enzymes is predominantly mediated by a redox-sensitive transcription factor NF-E2 related factor-2 (Nrf2). A variety of phytochemicals are able to activate Nrf2 thereby upregulating a set of enzymes including NADP(H): quinone oxidoreductase-1 (NQO1), superoxide dismutase (SOD), glutathione S-transferase (GST), hemeoxygenase-1 (HO-1), and glutamyl cysteine ligase (GCL). Nrf2 is sequestered in the cytoplasm as an inactive complex with its cytosolic repressor Kelch-like ECH associated protein 1 (Keap1). Dissociation of Nrf2 from the inhibitory protein Keap1 is a prerequisite for nuclear translocation and subsequent DNA binding of Nrf2. The genetic ablation of the Nrf2 results in severe airway inflammation and development of emphysema and asthma in mice. The importance of Nrf2 activation for chemoprevention was evident from a remarkably higher incidence of benzo[a]pyrene-induced gastric neoplasia in Nrf2-deficient mice, which were less responsive to the phase II enzyme inducer oltipraz than did wild-type mice. Thus, Nrf2 plays a central role in the regulation of constitutive and inducible expression of phase 2 enzymes in vivo.

Oxidative stress may also have a fundamental role in enhancing inflammation through the upregulation of redox-sensitive transcription factors, such as NF-κB and activating protein 1 (AP-1). Studies in macrophage cell lines and alveolar and bronchial epithelial cells show that oxidants cause the release of inflammatory mediators, such as IL-8, IL-1, and NO, and that these events are associated with increased expression of the genes for these inflammatory mediators and increased nuclear binding or activation of NF-κB. The linking of NF-κB to its consensus site in the nucleus leads to enhanced transcription of proinflammatory genes and therefore inflammation, which itself will produce more oxidative stress, creating a vicious circle of enhanced inflammation resulting from the increased oxidative stress. Therefore, it is believed that inhibitors of NF-κB and inhibitors of oxidative stress (activation of Nerf2) would work in concert and produce synergistic effects.

It is also believed that the presence of endothelin receptor and NF-κB antagonistic activity on the same molecule can be synergistic due to several reasons. First, reductions in endothelin levels due to the inhibition of gene transcription by NFκB will make inhibition of endothelin receptors more effective. Most endothelin receptor antagonists compete with endothelin for receptor binding; thus inhibition of endothelin receptor antagonists in the presence of reduced concentrations of endothelin should be enhanced substantially. Second, the effects of endothelin receptor antagonists and NF-κB antagonists on the apoptotic pathways complement each other. The inhibition of NF-κB induces apoptosis by regulating gene transcription of anti-apoptotic genes; whereas, endothelin acts as an antiapoptotic factor, modulating cell survival pathways through Bcl-2 and phosphatidylinositol 3-kinase/Akt pathways.

It would be highly desirable to enrich the fractions from clary sage roots that have activity against the NF-κB, Nerf2 and endothelin receptors, three medically important molecular targets. In particular, it would be desirable to provide the standardized root extract from clary sage for use in a pharmaceutical, nutraceutical and cosmeceuticals compositions suitable for the treatment and prevention of cancer, inflammation related diseases, cardiovascular diseases, including coronary heart disease, fungal and bacterial infections, as an anticoagulant, enhancement of adenylate cyclase activity, anti-inflammatory, vasodilator, antimyocardiac, retardation of cholesterol biosynthesis, inhibition of lipoprotein oxidation, treatment of acne and eczema, prevent hair loss, whitening skin and protect myocardium from hypoxia-induced cardiac contractile failure.

### BRIEF DESCRIPTION OF THE DRAWINGS

FIG. 1 is a graph of the identification of the active molecules from the Clary sage extract by LC-MS.

FIG. 2 is a graph or the stimulation of QR by SP001P and SP 2010 in mouse hepatoma Hepa 1c1c7 cells.

FIG. 3 is a graph of antioxidant activity of SP001P and SP2010 by ORAC Assay.

FIG. 4A is a graph of the inhibition of TNF-dependent kapaB-DNA binding in HeLa cells by SP 2010.

FIG. 4B is a graph of the inhibition of TNF-alpha mediated induction of luciferase activity in A549-NF-kapaB stable reporter cell line by SP 2010.

### SUMMARY OF THE INVENTION

The present invention relates to methods for the preparation of extracts from the roots of clary sage that are enriched in 3 compounds which together act synergistically. These compounds may inhibit oxidative stress, activation of NF-κB or endothelin receptor. The presence of the antagonistic activity towards oxidative stress, activation of NF-κB, and endothelin receptor in the same extract is synergistic and can lead to useful compositions suitable for the treatment and prevention of cancer, inflammation related diseases and cardiovascular diseases.

This invention relates to the use of certain novel compositions from clary sage (Salvia sclarea), which are inhibitors of activation of NF-κB, oxidative stress (activation of Nerf2) and inhibit the activity of the endothelin receptor that when administered together have a synergistic effect.

In particular, it relates to useful enriched fractions and pharmaceutical compositions from clary sage for use in the treatment of cardiovascular and inflammatory diseases and for cancers susceptible to an inhibitor of oxidative stress (activation of Nerf2), NF-κB inhibitor and an endothelin receptor inhibitor.

The present invention also relates to methods for preparing extracts and botanical drugs from clary sage so that they are enriched in the desired 3 components. The method includes assay guided steps that lead to the enrichment of several synergistic components from the roots of clary sage with activity against multiple medically useful molecular targets. These compounds inhibit oxidative stress (activation of Nerf2), activation of NF-κB or endothelin receptor. The simultaneous inhibition of activation of NF-κB, oxidative stress and endothelin receptor is synergistic and can lead to useful compositions suitable for the treatment and prevention of cancer, inflammation related diseases and cardiovascular diseases.

So in one embodiment the invention is an enriched fraction of clary sage which comprises an inhibitor of oxidative stress, an inhibitor of the endothelin receptor and an inhibitor of activation of NF-kB.

In yet another embodiment of the invention there is described a method for the treatment or prevention of cancer, inflammation, cardiovascular diseases, coronary heart disease comprising administering to a person in need of such treatment or prevention a therapeutically effective amount of an enriched fraction from clary sage which has been enriched for an inhibitor of oxidative stress, an inhibitor of the endothelin receptor and an inhibitor of activation of NF-kB.

The resulting isolated extract can be used in combination with a pharmaceutically acceptable carrier to provide a pharmaceutical composition suitable for use in treatment of a myriad of diseases. In one embodiment, the isolated extract can be used in the treatment of coronary heart disease.

### DETAILED DESCRIPTION OF THE INVENTION

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

The invention relates to methods for preparing extracts from clary sage that use assay-guided fractionation of the plant leading to the enrichment of compounds that inhibit oxidative stress, activation of NF-κB or endothelin receptor. The simultaneous inhibition of activation of NF-κB, oxidative stress and endothelin receptor is synergistic and can lead to useful compositions suitable for the treatment and prevention of cancer, inflammation related diseases and cardiovascular diseases.

The clary sage root, preferably after extraction of the essential oil and sclareol, is pulverized into a powder and is fractionated by a number of standard techniques. The inhibition of endothelin receptor, oxidative stress and activation of NF-κB by the plant extracts after each purification step is measured and the fractions with the enriched inhibitory activity are combined. The clary sage root is extracted with an organic solvent such as diethyl ether, petroleum ether, hexane, ethyl acetate, and methylene chloride and the extract is concentrated and washed with a water insoluble solvent such as sodium carbonate, sodium bicarbonate, and potassium hydroxide. The isolated extract is further fractionated by a myriad of standard techniques such as by solvent-solvent extraction, silica gel column chromatography, reverse phase liquid chromatography etc. to yield several fractions with different profiles of potent inhibitory activity against activation of NF-κB, oxidative stress (activation of Nerf2) and the activity of the endothelin receptor.

The assay-guided enrichment leads to plant extracts that are enriched in Tanshinone IIA, Tashinone I, Dihydrotanshinone, Cryptotanshinone, Hydroxytanshinone IIA, Methyl tanshinonate, Methylene tanshinone, Isotanshinone, Isotanshinone IIA, Isocryptotanshinone, Danshenxinhun A, B and C; compounds that have demonstrated inhibition of activation of NF-κB as disclosed in U.S. Application Ser. No. 60/779,142, hereby incorporated by reference.

The methods also lead to the enrichment of many abietane diterpenoids, apianane terpenoids, anthraquinone, and flavonoids that inhibit oxidative stress by the activation of the transcription factor called Nerf 2. Specifically, the plant fractions include carnosol, rosmanol, epirosmanol, isorosmanol, galdosol, rosmarinic acid, carnosic acid, miltirone, atuntzensin A, luteolin, 7-O-methyl luteolin, eupafolin, 12-O-methyl carnosol, hydroxycinnamic acid, caffeic acid, isoscutellarein 7-O-glucoside and genkwanin.

The fractions are also enriched in additional compounds that are known to inhibit endothelin receptors. These compounds include, but are not limited to, myriceric acid A (myriceron caffeoyl ester), myriceric acid B, myriceric acid C, and myriceric acid D.

The plant extracts described above may be formulated for administration in a pharmaceutical carrier in accordance with known techniques. See, e.g., Remington, The Science and Practice of Pharmacy (9^{th} Ed. 1995). In the manufacture of a pharmaceutical formulation according to the invention, the extract (including the physiologically acceptable salts thereof) is typically admixed with inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5 percent to 95 percent or 99 percent by weight of the extract.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated.

Formulations suitable for oral administration may be presented in discrete units, such as capsules, cachets, lozenges, or tables, each containing a predetermined amount of the extract; as a powder or granules; as a solution or a suspension in an aqueous or non-aqueous liquid; or as an oil-in-water or water-in-oil emulsion. Such formulations may be prepared by any suitable method of pharmacy which includes the step of bringing into association of the isolated extract and a suitable carrier (which may contain one or more accessory ingredients as noted above). In general, the formulations of the invention are prepared by uniformly and intimately admixing the isolated extract with a liquid or finely divided solid carrier, or both, and then, if necessary, shaping the resulting mixture. For example, a tablet may be prepared by compressing or molding a powder or granules containing the isolated extract, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the isolated extract in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the isolated extract, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by mixing the isolated extract with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)).

The therapeutically effective dosage of any isolated extract, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. The dosages will change for the particular condition being treated. For example, a dosage of from about 5 to 40 mg/kg may be suitable for treatment of coronary heart disease, but not for use as an antifungal agent.

As a general proposition, a preferred dosage from about 20 to 35 mg/kg is believed to have therapeutic efficacy, with all weights being calculated based upon the weight of the isolated extract, including the cases where a salt is employed. Toxicity concerns at the higher level may restrict intravenous dosages to a lower level such as up to about 20 mg/kg, with all weights being calculated based upon the weight of the active base, including the cases where a salt is employed. A preferred dosage from about 30 mg/kg to about 50 mg/kg may be employed for oral administration. Typically, a preferred dosage from about 20 mg/kg to 30 mg/kg may be employed for intramuscular injection. The duration of the treatment is usually once per day for a period of two to three weeks or until the condition is essentially controlled. The isolated extract of Clary Sage can be used to treat coronary heart disease or as an antifungal and/or antibacterial agent, as an anticoagulant, cholesterol retarding agent, acne, eczema, and myocardium from hypoxia-induced contractile failure.

The following examples are provided in order to further illustrate various embodiments of the invention and are not to be construed as limiting the scope thereof.

### EXAMPLES

Enrichment of inhibitors of NF-kB, oxidative stress (activation of Nerf2) and endothelin receptor A from clary sage root: Roots were collected from the ten-months-old clary sage plants after the aerial parts were harvested for its essential oil and sclareol. The roots were washed in tap water, air dried at 45° C. and ground in a Willey mill (10 u size particles). Three aliquots of 150 g of the powder were extracted with 1 L diethyl ether in a soxhlet extractor at 26° C. for 2 hr. The extract was washed with 5% sodium carbonate solution and then the solvent was removed on a rotary evaporator to obtain dried reddish-brown crude extract (8.5 grams). The crude extract was chromatographed on a flash column chromatography using silica gel.

Silica gel column chromatography: Medium pressure flash column chromatography was carried out using silica gel 60 (230-400 mesh). A glass column with a 30 mm diameter and 1000 mm length was packed with silica gel in hexane slurry. The crude sample (1 g) was loaded on to the column by adsorbing on to 1 g silica gel. The column was eluted with diethyl ether stepwise increasing portions of acetone in ether, stepwise increasing portions of methanol in acetone, and finally pure methanol. The flow rate of the eluents was maintained at 2 ml/min. All fractions were collected in 10 ml volume, concentrated to dryness in a savant concentrator. To aid in pooling different fractions, the activity against NF-kB, Nerf2 and endothelin receptor were determined. Further enrichment was carried out on a preparative thin layer chromatography.

Enrichment by high pressure liquid chromatography (HPLC). A HP 1090 HPLC with a DR-5 solvent delivery system and diode array detector. A 100×2.1 mm column (5 um Hypersil C18 reversed phase) was eluted with water-acetonitrile gradient with a flow rate of 0.6 ml/min. Fractions were concentrated to dryness in a savant concentrator and the activity against NF-kB, Nerf2 and endothelin receptor was determined.

Endothelin receptor antagonist activity (% inhibition) of Clary sage extracts

| Extract | 10 ug/ml | 100 ug/ml |
|---|---|---|
| Crude extract | 27.2 | 95.0 |
| First enrichment | 47.1 | 98.8 |
| Second enrichment | 84.7 | 99.4 |
| Third enrichment | 87.9 | 100.0 |

The fraction after third enrichment was designated as SP001P and subjected to fingerprinting using LC-MS analysis.

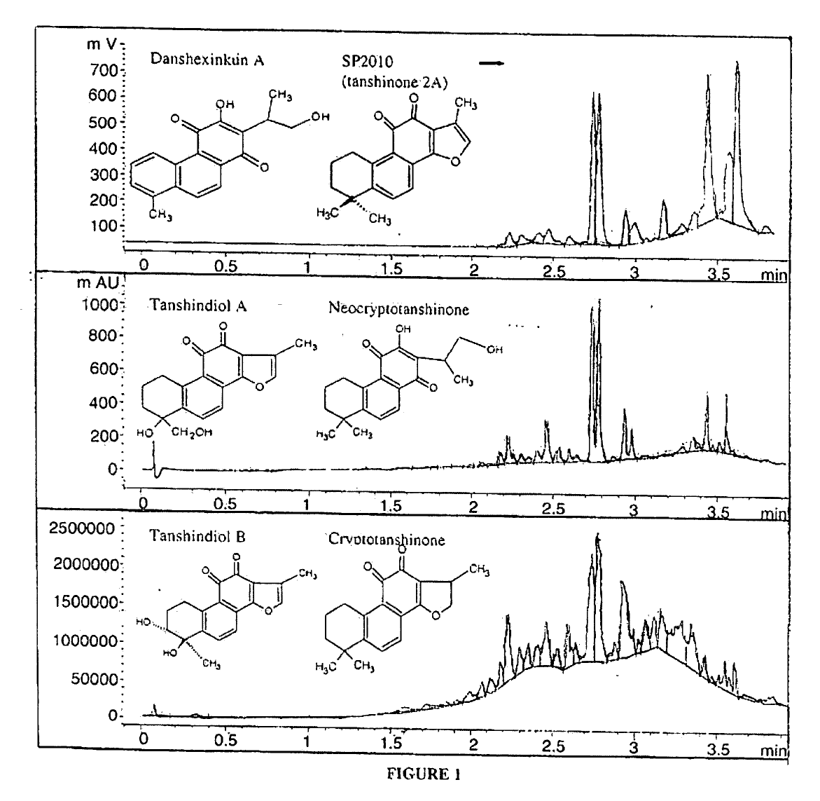

Identification of the active molecules from the Clary sage extract by LC-MS. The active Clary sage fraction SP001P was re-chromatographed on a hypersil C18 reversed phase column (100×2.1 mm, 5 μm), eluted with water-acetonitrile gradient with a flow rate of 0.6 ml/min. The sample was monitored based on the UV character and the fraction eluting at different times were collected, dried under nitrogen and finally freeze-dried to relatively pure compounds. Identification of the compound was confirmed by nuclear magnetic resonance (NMR) and mass spectra (MS). The NMR spectra were measured on a GE500 Omega spectrometer in CDCL3. Low resolution LIMS and EIMS were obtained using a Hewlett-Packard 5985-B mass spectrometer, and HRMS was determined on the A.E.I. MS-902. The structures of the compounds exhibiting NF-kB, Nerf2 and endothelin receptor inhibition have been identified based on their peak elution on the LC-MS profile (see FIG. 1). LC/MS mass spectra revealed an intense peak in the chromatogram at a mass of 2743 (FIG. 1). The compound was identified as Tanshinone IIA and was designated SP 2010. All other compounds were identified based on the mass, UV profile as well as by comparing to the published literature. About 20% of Tanshinone IIA (SP 2010) was detected in SP001P. Significant amount of other tanshinones including cryptotanshinone, neo-cryptotanshinone, tanshindiols and danshexinkun were also identified (FIG. 1).

The ability to rapidly identify novel or known compounds in plant extract is a critical factor in a natural products discovery program that is operating efficiently. The process to determine if the activity of a sample in a primary screen is due to a previously known substance is called de-replication. When interfaced with DAD, HPLC allows identification of known compounds by comparing their retention time and UV spectra to the compounds in a database. Structurally-defined set of natural product library of small molecule compounds with well defined UV absorption spectra to support the de-replication efforts has been used. Liquid chromatography in combination with mass spectrometry (LC-MS) has become a widely used tool for the de-replication of natural products. This method provides a rapid identification of compounds because molecular weight of a compound can be used as a search query in all databases. We employed LC-MS to determine the identity of the novel and known compounds in the crude fractions.

The structures of the compounds exhibiting activity have been identified based on their peak elution on the LC-MS profile (see FIG. 1). LC-MS was applied with multivariate statistics to finger print the ‘lead extracts’ that showed activities. The aim was to measure as comprehensively as possible the metabolite(s) finger printing of the extracts. These results contribute to a first step towards ultimate quality assurance and quality control (QA/QC) of the product. The biological activity always associated with more then one marker compound, most likely, by synergistic activity of the compounds present in the extract. As for a metabolite profiling approach, it is important to measure as many components as possible that show activity using the combination of LC-MS and other spectroscopy studies. This way it is possible to link the biological activity to the standardized extract.

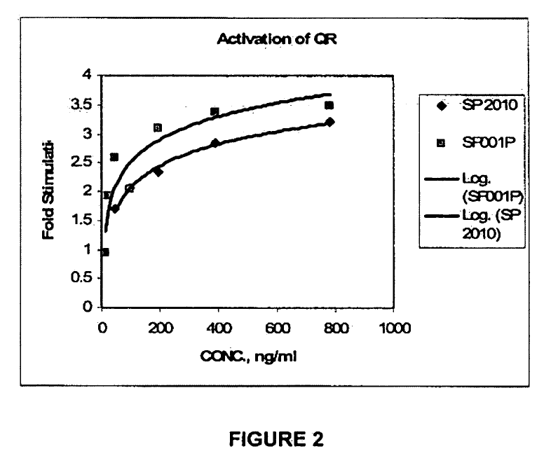

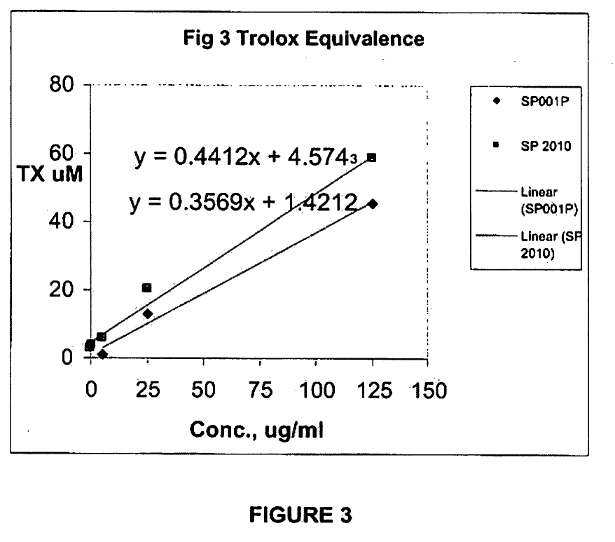

Activation of Quinone reductase (NAD[P]H:(quinone-acceptor) oxidoreductase (QR). We studied the effects of SP001P and SP 2010 on the activation of QR, a phase 2 enzyme in mouse hepatoma cells Hepa 1c1c7 (ATCC) at lower concentrations. QR protects cells against quinones and highly reactive semiquinones by catalyzing an obligate two-electron reduction of quinones to hydroquinones. We have developed a 96-well plate assay to assess QR activity in these cell lines. QR activity was measured by a menadione-coupled reduction of tetrazolium dye as modified from Prochaska and Santamari. Cells (10⁴ per well) were grown for 24 h and then exposed to serial dilutions of inducers for 24 h. Protein concentrations in a corresponding plate were determined by methylene blue staining as described earlier. SP001P and SP 2010 stimulated NAD[P]H:(quinone-acceptor) oxidoreductase (QR), a phase 2 enzyme in a dose dependent manner in mouse hepatoma Hepa 1c1c7 cells at concentrations known to inhibit B[a]P-induced transformation of RTE cells, (FIG. 2). The concentration required to double the specific activity of QR (CD value) for SP001P and SP 2010 were 24 ng/ml and 49 nM, respectively.

Antioxidant Capability of SP001P and SP 2010. The Oxygen Radical Absorbance Capacity (ORAC) assay depends on the free radical damage to a fluorescent probe, such as fluorescein, to result in a downward change of fluorescent intensity. The presence of antioxidants results in an inhibition in the free radical damage to the fluorescent compound. Reactions containing antioxidants and blanks (solvent) are run in parallel using equivalent amounts of a ROS generator and fluorescent probe. The area under the curve (AUC) from the experimental sample is calculated. Results from different samples are compared with Trolox®, (6-hydroxy-2,5,7,8-tetrametmethylchroman-2-carboxylic acid) a water soluble vitamin E analog and ORAC results are expressed as Trolox® equivalents. ORAC values are calculated using the regression equation between Trolox concentration and the net AUC and are expressed as micromole Trolox equivalents per gram of sample. SP001P and SP 2010 are potent antioxidants with values of 357 and 441 micromole Trolox equivalents per gram, respectively.

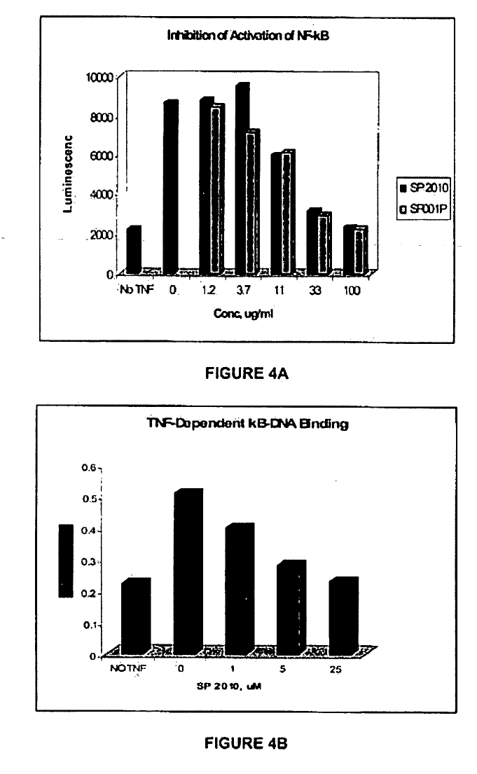

Inhibition of activation of NF-kB by SP001P and SP 2010. We evaluated the effects of SP001P and SP 2010 on the activity of NFκB transcription factor after activation with rhTNFα in a reporter cell line A549/NFκB-luc designed by Panomics according to the manufacturer's instructions. Results are shown in FIG. 4A; a dose-dependent decrease in transactivation of NF-κB by SP001P and SP 2010 was observed (FIG. 4A). It is interesting to note that although SP001P has only 20% of SP 2010 yet it produced a dose response curve that was very similar with SP 2010 (FIG. 4A). These data suggest that SP 2010 and other components in the Clary Sage extract contribute towards its inhibitory activity. We studied the effects of SP 2010 on TNF-α-dependent activation of NF-κB binding to KB site in nuclear extracts of HeLa cells. To measure the binding of the activated NF-κB to its consensus KB sequence, nuclear extracts from HeLa cells treated with SP 2010 and stimulated with TNFα were prepared by the Panomics Nuclear Extraction kit. Nuclear extracts were diluted to 2 μg/μl and 10 μl of the extract was used to quantify transcription factor activation, specifically that of NF-κB p50 (ELISA, Panomics). Results are shown in FIG. 4B. A dose-dependent decrease in NF-κB binding to KB site in HeLa cells by SP 2010 was observed (FIG. 4B).

That which is claimed:

1. An enriched fraction of clary sage which comprises an inhibitor of oxidative stress, an inhibitor of the endothelin receptor and an inhibitor of activation of NF-kB.

2. An enriched fraction according to claim 1, wherein the inhibitor of activation of NF-kB is enriched from the group consisting of Tanshinone IIA, Tashinone I, Dihydrotanshinone, Cryptotanshinone, Hydroxytanshinone IIA, Methyl tanshinonate, Methylene tanshinone, Isotanshinone, Isotanshinone IIA, Isocryptotanshinone, Danshenxinhun A, B and C.

3. An enriched fraction according to claim 1, wherein the inhibitor of activation of oxidative stress is enriched from the group consisting of carnosol, rosmanol, epirosmanol, isorosmanol, galdosol, rosmarinic acid, carnosic acid, miltirone, atuntzensin A, luteolin, 7-O-methyl luteolin, eupafolin, 12-O-methyl carnosol, hydroxycinnamic acid, caffeic acid, isoscutellarein 7-O-glucoside and genkwanin.

4. An enriched fraction according to claim 1, wherein the inhibitor of endothelin receptor is enriched from the group consisting of myriceric acid A (myriceron caffeoyl ester), myriceric acid B, myriceric acid C, and myriceric acid D.

5. A composition according to claim 1 which further comprises a pharmaceutically acceptable carrier.

6. A method for the treatment or prevention of a disease selected from the group consisting of cancer, inflammation, cardiovascular diseases, coronary heart disease comprising administering to a person in need of such treatment or prevention a therapeutically effective amount of an enriched fraction from clary sage which has been enriched for an effective amount of an inhibitor of oxidative stress, an inhibitor of the endothelin receptor and an inhibitor of activation of NF-kB.
